(19) United States
(12) Patent Application Publication
(10) Pub. No.: US 2025/0041308 A1
(43) Pub. Date: Feb. 6, 2025

(54) BRUTON'S TYROSINE KINASE INHIBITORS AS ANTIVIRAL AGENTS

(71) Applicant: University of Florida Research Foundation, Incorporated, Gainesville, FL (US)

(72) Inventors: Michael MCINTOSH, Gainesville, FL (US); Sumita BHADURI-MCINTOSH, Gainesville, FL (US)

(21) Appl. No.: 18/718,478

(22) PCT Filed: Jan. 5, 2023
(86) PCT No.: PCT/US2023/060140

§ 371 Date: Jun. 11, 2024

Related U.S. Application Data
(60) Provisional application No. 63/266,526, filed on Jan. 7, 2022.

(51) Int. Cl. A61K 31/519; A61K 31/4985; A61P 31/22

(52) U.S. Cl. CPC A61K 31/519; A61K 31/4985; A61P 31/22

(57) ABSTRACT

Use of pharmaceutical compositions comprising Bruton's tyrosine kinase (BTK) inhibitors in decreasing Epstein-Barr Virus lytic cycle or switch from latency to lytic cycle are described. The pharmaceutical compositions can be used to prevent or reduce the risk of developing an EBV-positive lymphoproliferative disease (LPD). EBV-associated malignancy, or pre-malignancy, including in immunosuppressed subjects.

### CROSS-REFERENCE TO RELATED APPLICATIONS

This application claims the benefit of U.S. Provisional Application No. 63/266,526, filed Jan. 7, 2022, which is incorporated herein by reference

### INTRODUCTION

The Epstein-Barr virus (EBV) is a human herpesvirus and is one of the most common viruses in humans. EBV infects B cells of the immune system and epithelial cells. Once EBV's initial lytic infection is brought under control, EBV latency persists in the individual's B cells for the rest of their life.

Most people become infected with EBV and gain adaptive immunity. In the United States, about half of all five-year-old children and about 90% of adults have evidence of previous infection.

EBV is associated with various non-malignant, premalignant, and malignant lymphoproliferative diseases such as Burkitt lymphoma, diffuse large B-cell lymphoma (DLBCL), hemophagocytic lymphohistiocytosis, and Hodgkin's lymphoma; non-lymphoid malignancies such as gastric cancer and nasopharyngeal carcinoma; and conditions associated with human immunodeficiency virus such as hairy leukoplakia and central nervous system lymphomas (Kosowicz et al., J. Virology 2017. “Drug Modulators of B Cell Signaling Pathways and Epstein-Barr Virus Lytic Activation”).

EBV has two modes of infection: latent and lytic. The lytic cycle, or productive phase, results in the production of infectious virions. That said, EBV infection is predominantly latent and malignancies can arise from latently infected cells. The switch from latency to the lytic cycle is known as EBV (re) activation. Switch to lytic cycle can also increase the likelihood of lymphoproliferative disease. The viral lytic cycle may contribute to the development and maintenance of malignancies through increasing the pool of latently infected cells, the induction of growth factors, and oncogenic cytokine production. Immunosuppressed patients are particularly at risk of developing EBV+lymphoproliferative disorders.

### SUMMARY

Described are methods of inhibiting EBV switch from latency to lytic cycle, or EBV reactivation, in a subject comprising administering to the subject an effective dose of a Bruton's tyrosine kinase (BTK) inhibitor or a pharmaceutical composition comprising a BTK inhibitor. Is it known that EBV switch to lytic cycle can contribute to lymphoproliferative disease. Therefore, inhibiting the switch to lytic cycle (i.e., reactivation) can be used to prevent or reduce the risk of developing an EBV-positive (EBV″) lymphoproliferative disease (LPD).

BTK inhibitors useful in the methods include both irreversible BTK inhibitors and reversible BTK inhibitors. BTK inhibitors include, but are not limited to, ibrutinib, acalabrutinib, pirtobrutinib, zanubrutinib, tirabrutinib, evobrutinib, vecabrutinib, tolebrutinib, elsubrutinib, nemtabrutinib, fenebrutinib, rilzabrutinib, remibrutinib, Poseltinib, M7583, Orelabrutinib, Spebrutinib, Vecabrutinib, TL-895, Edralbrutinib, Luxeptinib, Spebrutinib, and HM71224.

Lymphoproliferative diseases that can be prevented, or for which the risk can be reduced, using the described methods include, but are not limited to, iatrogenic lymphoproliferative disorder, post-transplant lymphoproliferative disorder (PTLD), Burkitt lymphoma, B-cell non-Hodgkin lymphoma, diffuse large B-cell lymphoma (DLBCL), CNS lymphoma, NK/T-cell lymphoma, T cell non-Hodgkin lymphoma, Hodgkin lymphoma, and AIDS-related lymphoma.

The described methods can be used to inhibit EBV switch from latency to lytic cycle and/or to prevent or reduce the risk of developing an EBV″ LPD in an immunosuppressed subject. Immunosuppressed subjects are more susceptible to LPD and so are suitable subjects for prophylactic treatment with BTK inhibitors. Subjects suitable for treatment with the described methods include subjects having an autoimmune disorder, such as lupus, systemic lupus erythematosus, rheumatoid arthritis, myelodysplastic syndromes (MDS), inflammatory bowel disease, Crohn's disease, ulcerative colitis, multiple sclerosis, aplastic anemia, alopecia areata, or HIV/AIDS, or a subject being treating with immunosuppressive or immunomodulatory therapy, such as T cell suppressive therapy or anti-TNF alpha therapy. T cell suppressive therapy can be, but is not limited to, antithymocyte globulin or recombinant antithymocyte globulin (ATG or rATG) therapy.

In some embodiments, the described methods are used to prophylactically treat an organ transplant recipient or a hematopoietic transplant recipient. In some embodiments, the organ transplant recipient or the hematopoietic transplant recipient is seronegative for Epstein-Barr virus (EBV) at the time of the transplant, has an active or suspected active EBV infection at the time of or following the transplant, is EBV positive and receives an EBV-negative donor organ or hematopoietic transplant, has a high degree of HLA mismatch with the donor, has an active or suspected active CMV infection at the time of the transplant, is CMV positive and receives a CMV-negative donor organ or hematopoietic transplant, has or is suspected of having acute or chronic graft versus host disease, is at risk of acute or chronic graft versus host disease, suffers from or is suspected of suffering from T-cell depletion, has diabetes, or is on an intense immunosuppressive drug regimen, or combination of one or more of the above.

Prophylactic treatment with a pharmaceutical composition comprising an effective dose of a BTK inhibitor (BTK inhibitor therapy), comprises administering the BTK inhibitor therapy prior to diagnosis of an LPD. For a transplant patient, the BTK inhibitor therapy can be initiated prior to the transplant, at the time of the transplant, or within about 2 weeks to about 8 week of the transplant. Similarly, to a subject on immunosuppressive or immunomodulatory therapy, including transplant patients, the BTK inhibitor therapy can be initiated prior to the start of the transplant immunosuppressive or immunomodulatory therapy, concurrent with the start of the immunosuppressive or immunomodulatory therapy, or within about 2 weeks to about 8 weeks of the start of the immunosuppressive or immunomodulatory therapy. Prophylactic treatment with BTK inhibitor therapy can be continued for a period of about 1 year to about 2 years, or longer.

In some embodiments, EBV levels in the subject are monitored and the pharmaceutical composition containing the BTK inhibitor is administered to the subject if the levels of EBV in the subject increase.

Also described are methods of treating oral hairy leukoplakia (OHL) in a subject having HIV/AIDS, the methods comprising administering to the subject a pharmaceutical composition comprising an effective dose of a Bruton's tyrosine kinase (BTK) inhibitor. The pharmaceutical composition can be administered to the subject upon diagnosis of OHL and can be continued for as long as the OHL condition persists.

Described are methods of decreasing cleavage of procaspase 1 to caspase 1 in EBV infected cells in a subject comprising administering to the subject a pharmaceutical composition comprising an effective dose of a Bruton's tyrosine kinase (BTK) inhibitor. Cleavage of procaspase 1 to caspase 1 is a marker of inflammasome activation. Decreasing cleavage of procaspase 1 to caspase 1 can lead reduced reactivation of EBV and or reduced risk of development of EBV lymphoproliferative diseases. Thus, in some embodiments, administering to the subject an effective dose of a Bruton's tyrosine kinase (BTK) inhibitor or a pharmaceutical composition comprising a BTK inhibitor results in decreasing reactivation of EBV and risk of development of EBV lymphoproliferative diseases in the subject. BTK inhibitors useful in the methods include both irreversible BTK inhibitors and reversible BTK inhibitors. BTK inhibitors include, but are not limited to, ibrutinib, acalabrutinib, zanubrutinib, pirtobrutinib, tirabrutinib, evobrutinib, vecabrutinib, tolebrutinib, elsubrutinib, nemtabrutinib, fenebrutinib, rilzabrutinib, remibrutinib, Poseltinib, M7583, Orelabrutinib, Spebrutinib, Vecabrutinib, TL-895, Edralbrutinib, Luxeptinib, Spebrutinib, and HM71224.

### BRIEF DESCRIPTION OF THE DRAWINGS

FIG. 1. Western blot to detect expression of EA-D (EBV Early antigen-diffuse) and EBV immediate early protein ZEBRA in HH514-16 Burkitt lymphoma cells following 2 hour pretreatment with ibrutinib and 24 hour exposure to the EBV lytic cycle inducing agent sodium butyrate (NaB, a histone deacetylase inhibitor).

FIG. 2. Western blot of EA-D and ZEBRA expression in HH514-16 Burkitt lymphoma cells following 2 hour pretreatment with ibrutinib and 24 hour exposure to the EBV lytic cycle inducing agent AZA (5-aza-2′-deoxycytidine, a DNA methyltransferase inhibitor).

FIG. 3. Western blot of EA-D and ZEBRA expression in Burkitt lymphoma Akata cells following 2 hour pretreatment with ibrutinib and 24 hour exposure to the EBV lytic cycle inducing agent rabbit-anti human IgG.

FIG. 4. Western blot showing ZEBRA expression in HH514-16 Burkitt lymphoma cells following 2 hour pretreatment with Zanubrutinib, Acalabrutinib, or Ibrutinib and 24 hour exposure to the EBV lytic cycle inducing agent sodium butyrate (NaB, a histone deacetylase inhibitor).

FIG. 5. Flow cytometry plots showing ZEBRA expression (lytic) or isotype-matched control antibody (isotype) in Mutu-I Burkitt lymphoma cells following 2 hour pretreatment with ibrutinib and 24 hour exposure to the EBV lytic cycle inducing agent sodium butyrate (NaB, a histone deacetylase inhibitor).

FIG. 6. Flow cytometry plots showing ZEBRA expression (lytic) or isotype-matched control antibody (isotype) in Mutu-I Burkitt lymphoma cells following 2 hour pretreatment with Zanubrutinib or Acalabrutinib and 24 hour exposure to the EBV lytic cycle inducing agent sodium butyrate (NaB, a histone deacetylase inhibitor).

FIG. 7. Western blot showing ZEBRA, Procaspase 1, cleaved caspase 1, and β-actin (control) in Mutu-I Burkitt lymphoma cells following 2 hour pretreatment with Zanubrutinib, Acalabrutinib, or Ibrutinib and 24 hour exposure to the EBV lytic cycle inducing agent sodium butyrate (NaB, a histone deacetylase inhibitor).

### DETAILED DESCRIPTION

### I. Definitions

Before describing the present teachings in detail, it is to be understood that the disclosure is not limited to specific compositions or process steps, as such may vary. As used in this specification and the appended claims, the singular form “a,” “an,” and “the” include plural references unless the context clearly dictates otherwise. Thus, for example, reference to “a peptide” includes a plurality of peptides and the like. The conjunction “or” is to be interpreted in the inclusive sense, i.e., as equivalent to “and/or,” unless the inclusive sense would be unreasonable in the context.

The use of “comprise,” “comprises,” “comprising,” “contain,” “contains,” “containing,” “include,” “includes,” and “including” are not intended to be limiting. It is to be understood that both the foregoing general description and detailed description are exemplary and explanatory only and are not restrictive of the teachings. To the extent that any material incorporated by reference is inconsistent with the express content of this disclosure, the express content controls.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 0 to 20%, 0 to 10%, 0 to 5%, or up to 1% of a given value. Where particular values are described in the application and claims, unless otherwise stated the term “about” meaning within an acceptable error range for the particular value should be assumed.

All ranges are to be interpreted as encompassing the endpoints in the absence of express exclusions such as “not including the endpoints”; thus, for example, “within 10-15” includes the values 10 and 15. One skilled in the art will understand that the recited ranges include the end values, as whole numbers in between the end values, and where practical, rational numbers within the range (e.g., the range 5-10 includes 5, 6, 7, 8, 9, and 10, and where practical, values such as 6.8, 9.35, etc.). When values are expressed as approximations, by use of the antecedent “about,” it will be understood that the particular value forms a further aspect. For example, if the value “about 10” is disclosed, then “10” is also disclosed.

“Subject” refers to an animal, such as a mammal, for example a human. The methods described herein can be useful in both humans and non-human animals. In some embodiments, the subject is a mammal (such as an animal model of disease). Mammal includes, without limitation, mice, rats, rabbits, guinea pigs, dogs, cats, sheep, goats, cows, horses, primates, such as monkeys, chimpanzees, and apes, and humans. In some embodiments, the subject is human.

The terms “treat,” “treatment,” and the like, mean the methods or steps taken to prevent or provide relief from or alleviation of the number, severity, and/or frequency of one or more symptoms of a disease or condition in a subject. The terms “treating,” “treatment,” “therapeutic,” or “therapy” do not necessarily mean total cure or abolition of the disease or condition. Any alleviation of any undesired signs or symptoms of a disease or condition, to any extent can be considered treatment and/or therapy.

An “effective amount” refers to an amount that is capable of treating or ameliorating a disease or condition or otherwise capable of producing an intended therapeutic effect.

A “pharmacologically effective amount,” “therapeutically effective amount,” “effective amount,” or “effective dose” refers to that amount of an agent to produce the intended pharmacological, therapeutic, or preventive result, such as, but not limited to, treating or ameliorating a disease or condition.

“Immunosuppressive therapy” and “immunomodulatory therapy” are drug regimens that patients use to lower or modulate the activity of their immune system. The drug regiments prevent the immune system from overreacting and rejecting or damaging transplanted organs and tissues. Most transplant patients receive immunosuppressive therapy or immunomodulatory therapy when receiving a transplant. Immunosuppressive therapy and immunomodulatory therapy may also be used to treat conditions in which the immune system is overactive, such as autoimmune diseases and allergies. Some types of immunosuppressive therapy may increase a person's risk of cancer by lowering the body's ability to kill cancer cells

### II. Inhibition of EBV Lytic Cycle and Cancer Prevention.

Described is the use of a Bruton's tyrosine kinase (BTK) inhibitor or a pharmaceutical composition comprising a BTK inhibitor to inhibit Epstein-Barr virus (EBV) switch from latency to lytic cycle, prevent or reduce B-cell infection by EBV, or inhibit EBV reactivation, in a subject. EBV reactivation is understood to cause EBV LPDs. Treatment of B cells harboring latent EBV, or a subject with latent EBV infection, with BTK inhibitors reduces EBV switch to lytic cycle and reduces the production of virus particles needed to support cancer development. Thus, inhibiting the EBV switch to lytic cycle can be used to prevent or reduce the risk of developing an EBV⁺ LPD.

Bruton's tyrosine kinase (abbreviated Btk or BTK), also known as tyrosine-protein kinase BTK, is a tyrosine kinase that is encoded by the BTK gene in humans. Additional aliases include ATK, PSCTK1, AGMX1, XLA, Bruton Agammaglobulinemia Tyrosine Kinase, Tyrosine-Protein BTK, Bruton's Tyrosine Kinase, B-cell Progenitor Kinase, IMD1, BPK, Dominant-Negative Kinase-Deficient Bruton's Tyrosine Kinase, IGHD3, AT, EC 2.7.10.2, or EC 2.7.10. BTK plays a role in B cell development. BTK is required for transmitting signals from the pre-B cell receptor that forms after successful immunoglobulin heavy chain rearrangement. It also has a role in mast cell activation through the high-affinity IgE receptor. BTK contains a PH domain that binds phosphatidylinositol (3,4,5)-trisphosphate (PIP3). PIP3 binding induces BTK to phosphorylate phospholipase C, which in turn hydrolyzes PIP2, a phosphatidylinositol, into two second messengers, inositol triphosphate (IP3) and diacylglycerol (DAG), which then go on to modulate the activity of downstream proteins during B-cell signaling.

BTK also plays a role in activating the major cellular inflammatory machinery called the inflammasome. Upon activation, the inflammasome causes activation and release of chemicals called cytokines that cause inflammation and fever. This is often a response to viral infections. While the inflammasome is a defense mechanism against viruses, some viruses such as the Epstein-Barr virus (EBV) can exploit it to trigger viral replication, which can be detrimental to the host (Burton et al., Proceedings of the National Academy of Sciences 2020. “A promiscuous inflammasome sparks replication of a common tumor virus”). Without wishing to be limited by theory, administration of a BTK inhibitor may block activation of the inflammasome which in turn blocks reactivation of EBV, resulting in a decrease in lytic viral replication and formation of EBV″ LPD. Administering a BTK inhibitor to a subject, prevents or reduces the risk of the subject developing an EBV LPD.

Immunosuppressed subjects are susceptible to LPD and so are suitable subjects for prophylactic treatment with BTK inhibitors. Immunosuppressed subjects include, but are not limited to, subjects on immunosuppression or immunomodulation therapy and subjects suffering from an autoimmune disorder or disease. An immunosuppressed subject may be any patient that has undergone T cell immunosuppression or is currently on T cell immunosuppression.

The described methods can be used to inhibit activation of EBV or inhibit switching of EBV from latent to lytic phase in a subject at risk of developing an LPD. In some embodiments, the subject is an immunosuppressed subject. In some embodiments, the subject has been on or is currently on immunosuppressive therapy or immunomodulatory therapy. In some embodiments, the immunosuppressed subject is an organ transplant recipient or a hematopoietic transplant recipient. In some embodiments, the subject is an HIV/AIDS patient. In some embodiments, the subject has an autoimmune condition. The autoimmune condition can be, but is not limited to lupus, systemic lupus erythematosus, rheumatoid arthritis, myelodysplastic syndromes (MDS), inflammatory bowel disease, Crohn's disease, ulcerative colitis, multiple sclerosis, aplastic anemia, or alopecia areata. The LPD or EBV-associated malignancy or pre-malignancy can be, but is not limited to, iatrogenic lymphoproliferative disorder, post-transplant lymphoproliferative disorder (PTLD), Burkitt lymphoma, B-cell non-Hodgkin lymphoma, diffuse large B-cell lymphoma (DLBCL), CNS lymphoma, NK/T-cell lymphoma, T cell non-Hodgkin lymphoma, Hodgkin lymphoma, nasopharyngeal cancer, gastric carcinoma, oral hairy leukoplakia (in HIV positive patients), and an AIDS-related lymphomas.

In some embodiments, EBV levels in the subject are monitored and the pharmaceutical composition containing the BTK inhibitor is administered to the subject if the levels of EBV in the subject increase.

Described are methods of preventing or reducing the risk of developing an EBV″ LPD in a subject comprising administering to the subject a pharmaceutical composition comprising an effective dose of a BTK inhibitor. In some embodiments, the subject is an immunosuppressed subject. In some embodiments, the subject has been on or is currently on immunosuppressive therapy or immunomodulatory therapy. In some embodiments, the immunosuppressed subject is an organ transplant recipient or a hematopoietic transplant recipient. In some embodiments, the subject is an HIV/AIDS patient. In some embodiments, the subject has an autoimmune condition. The autoimmune condition can be, but is not limited to lupus, systemic lupus erythematosus, rheumatoid arthritis, myelodysplastic syndromes (MDS), inflammatory bowel disease, Crohn's disease, ulcerative colitis, multiple sclerosis, aplastic anemia, or alopecia areata. The lymphoproliferative disease or EBV-associated malignancy or pre-malignancy can be, but is not limited to, post-transplant lymphoproliferative disorder (PTLD), Burkitt lymphoma, B-cell non-Hodgkin lymphoma, diffuse large B-cell lymphoma (DLBCL), CNS lymphoma, NK/T-cell lymphoma, gastric carcinoma, T cell non-Hodgkin lymphoma, Hodgkin lymphoma, nasopharyngeal cancer, oral hairy leukoplakia (in HIV positive patients), and an AIDS-related lymphomas. The described pharmaceutical compositions are administered to an immunosuppressed subject prior to diagnosis of an LPD.

Described are methods of preventing or reducing the risk of developing an EBV-positive LPD or EBV-associated malignancy or pre-malignancy in an immunosuppressed subject comprising administering to the subject a pharmaceutical composition comprising an effective dose of a Bruton's tyrosine kinase (BTK) inhibitor. Lymphoproliferative diseases, malignancies, or pre-malignancies that can be prevented or for which the risk can be reduced include, but are not limited to, iatrogenic lymphoproliferative disorder, post-transplant PTLD, Burkitt lymphoma, B-cell non-Hodgkin lymphoma, diffuse large B-cell lymphoma (DLBCL), CNS lymphoma, NK/T-cell lymphoma, T cell non-Hodgkin lymphoma, Hodgkin lymphoma, nasopharyngeal cancer, gastric carcinoma, oral hairy leukoplakia (in HIV positive patients), and an AIDS-related lymphomas. An immunosuppressed subject may be on immunosuppressive or immunomodulatory therapy. An immunosuppressed subject may be any patient that has undergone T cell immunosuppression or is currently on T cell immunosuppression. Immunosuppressive or immunomodulatory therapy includes, but is not limited to, T cell suppressive therapy or anti-TNF alpha therapy. T cell suppressive therapy can be, but is not limited to, antithymocyte globulin or recombinant antithymocyte globulin (ATG or rATG) therapy. The described pharmaceutical compositions are administered to an immunosuppressed subject prior to diagnosis of an LPD.

Immunosuppressive or immunomodulatory drugs include, but are not limited to, corticosteroids (e.g., prednisone (Deltasone, Orasone), budesonide (Entocort EC), prednisolone (Millipred)), Janus kinase inhibitors (e.g., tofacitinib (Xeljanz)), calcineurin inhibitors (e.g., cyclosporine (Neoral, Sandimmune, SangCya), tacrolimus (Astagraf XL, Envarsus XR, Prograf)), mTOR inhibitors (e.g., sirolimus (Rapamune), everolimus (Afinitor, Zortress), inosine monophosphate dehydrogenase (IMDH) inhibitors (e.g., azathioprine (Azasan, Imuran), leflunomide (Arava), mycophenolate (CellCept, Myfortic), biologics (e.g., abatacept (Orencia), adalimumab (Humira), anakinra (Kineret), certolizumab (Cimzia), etanercept (Enbrel), golimumab (Simponi), infliximab (Remicade), ixekizumab (Taltz), natalizumab (Tysabri), rituximab (Rituxan), secukinumab (Cosentyx), tocilizumab (Actemra), ustekinumab (Stelara), vedolizumab (Entyvio), monoclonal antibodies (e.g., basiliximab (Simulect), daclizumab (Zinbryta)), and antimetabolites (e.g., methotrexate).

Administration of a BTK inhibitor to an immunosuppressed subject to prevent or reduce the risk of developing LPD can be initiated prior initiated prior to the start of the immunosuppressive or immunomodulatory therapy, concurrent with the start of the immunosuppressive or immunomodulatory therapy, or within about 1 week, within about 2 weeks, within about 3 weeks, withing about 4 weeks, within about 2 month, within about 5 weeks, within about 6 weeks, withing about 7 weeks, within about 8 weeks, within about 2 months, within about 3 months, or within about 2 years of the start of the immunosuppressive or immunomodulatory therapy. In some embodiments, the administration of the BTK inhibitor to the subject is initiated on the same day as the immunosuppressive or immunomodulatory therapy, or on about day 1, on about day 2, on about day 3, on about day 4, on about day 5, on about day 6, on about day 7, on about day 8, on about day 9, on about day 10, on about day 11, on about day 12, on about day 13, on about day 14, on about day 15, on about day 16, on about day 17, on about day 18, on about day 19, on about day 20, or on about day 21 after the start of the immunosuppressive or immunomodulatory therapy. In some embodiments, the administration of the BTK inhibitor to the subject is initiated within about two weeks of the start of the immunosuppressive or immunomodulatory therapy. In some embodiments, EBV levels are monitored in the subject and the BTK inhibitor is administered by the subject if a spike or increase in EBV levels is observed. EBV levels can be monitored using methods and tests available in the art. The administration of a BTK inhibitor can be continued for a period of about 1 year to about 2 years, or longer. The administration of a BTK inhibitor can be continued for as long as the subject is on immunosuppressive or immunomodulatory therapy. The BTK inhibitor is administered to an immunosuppressed subject that has not been diagnosed with a LPD or B cell lymphoma.

In some embodiments, the described methods can be used to prevent or reduce the risk of developing EBV″ LPD in a subject that has been on or is currently on T cell suppressive therapy or anti-TNF alpha therapy. The T cell suppressive therapy can be, but is not limited to, antithymocyte globulin or recombinant antithymocyte globulin therapy.

Described are methods of preventing or reducing the risk of developing an EBV-positive lymphoproliferative disease (LPD), malignancy, or pre-malignancy in a transplant recipient comprising administering to the recipient a pharmaceutical composition comprising an effective dose of a BTK inhibitor. Lymphoproliferative diseases that can be prevented or for which the risk can be reduced include, but are not limited to, iatrogenic lymphoproliferative disorder, post-transplant lymphoproliferative disorder (PTLD), Burkitt lymphoma, B-cell non-Hodgkin lymphoma, diffuse large B-cell lymphoma (DLBCL), CNS lymphoma, NK/T-cell lymphoma, T cell non-Hodgkin lymphoma, Hodgkin lymphoma, nasopharyngeal cancer, or gastric cancer. The transplant can be an organ transplant or a hematopoietic transplant.

In some embodiments, the methods can be used in the post-transplant period to protect transplant recipients developing EBV+PTLD/LPD (post-transplant lymphoproliferative disease). The described methods can be used to prophylactically treat organ transplant recipients or a hematopoietic transplant recipients. In some embodiments, the organ transplant recipient or the hematopoietic transplant recipient is under 20 years of age, is seronegative for Epstein-Barr virus (EBV) at the time of the transplant, has an active or suspected active EBV infection at the time of or following the transplant, is EBV positive and receives an EBV-negative donor organ or hematopoietic transplant, has a high degree of HLA mismatch with the donor, has an active or suspected active CMV infection at the time of the transplant, is CMV positive and receives a CMV-negative donor organ or hematopoietic transplant, has or is suspected of having acute or chronic graft versus host disease, is at risk of acute or chronic graft versus host disease, suffers from or is suspected of suffering from T-cell depletion, has diabetes, or is on an intense immunosuppressive drug regimen, or combination of one of more of the above. The EBV-PTLD/LPD and/or EBV-cancer can be, but is not limited to, iatrogenic lymphoproliferative disorder, post-transplant lymphoproliferative disorder (PTLD), Burkitt lymphoma, B-cell non-Hodgkin lymphoma, diffuse large B-cell lymphoma (DLBCL), CNS lymphoma, NK/T-cell lymphoma, T cell non-Hodgkin lymphoma, Hodgkin lymphoma, and nasopharyngeal cancer. In some embodiments, the BTK inhibitor is administered to a transplant recipient that has not been diagnosed with a LPD or B cell lymphoma.

Prophylactic treatment with a pharmaceutical composition comprising an effective dose of a BTK inhibitor (BTK inhibitor therapy), comprises administering the BTK inhibitor therapy prior to diagnosis of an LPD. For a transplant patient, the BTK inhibitor therapy can be initiated prior to the transplant, at the time of the transplant, or within about 2 weeks to about 8 week of the transplant. Prophylactic treatment with BTK inhibitor therapy can be continued for a period of about 1 year to about 2 years, or longer.

The risk of EBV-LPD is highest in the first 2 years following transplantation. In some embodiments, the administration of the BTK inhibitor to the subject is initiated within about two weeks of the transplant and is continued for about 2 years.

Currently, the standard of care for transplant recipients is to follow EBV loads in blood at regular intervals and treat with Rituximab if EBV load starts rising. Rituximab indiscriminately destroys all B cells regardless of whether they are infected with EBV, often causing long term global B cell immunodeficiency. The described methods can be used to eliminate, delay, or reduce the need for Rituximab therapy.

Administration of a BTK inhibitor to a subject that is an organ transplant recipient or a hematopoietic transplant recipient to prevent or reduce the risk of developing EBV-PTLD/LPD can be initiated prior to the transplant, at the time of the transplant, within about 1 week of the transplant, within about 2 weeks of the transplant, within about 3 weeks of the transplant, withing about 4 weeks of the transplant, within about 2 month of the transplant, within about 5 weeks of the transplant, within about 6 weeks of the transplant, withing about 7 weeks of the transplant, within about 8 weeks of the transplant, within about 2 months of the transplant, within about 3 months of the transplant, or within about 2 years of the transplant. In some embodiments, the administration of the BTK inhibitor to the subject is initiated of the day of the transplant, on about day 1 after the transplant, on about day 2 after the transplant, on about day 3 after the transplant, on about day 4 after the transplant, on about day 5 after the transplant, on about day 6 after the transplant, on about day 7 after the transplant, on about day 8 after the transplant, on about day 9 after the transplant, on about day 10 after the transplant, on about day 11 after the transplant, on about day 12 after the transplant, on about day 13 after the transplant, on about day 14 after the transplant, on about day 15 after the transplant, on about day 16 after the transplant, on about day 17 after the transplant, on about day 18 after the transplant, on about day 19 after the transplant, on about day 20 after the transplant, or on about day 21 after the transplant. In some embodiments, the administration of the BTK inhibitor to the subject is initiated within about two weeks of the transplant. In some embodiments, EBV levels are monitored in the subject and the BTK inhibitor is administered by the subject is a spike or increase in EBV levels is observed. EBV levels can be monitored using methods and tests available in the art.

A BTK inhibitor can be administered to a subject that is an organ transplant recipient or a hematopoietic transplant recipient to prevent or reduce the risk of developing EBV-PTLD/LPD for a period of about 1 to about 5 years, about 1 to about 4 years, about 1 to about 3 years, or about 1 to about 2 years. In some embodiments, the BTK inhibitor is administered to the subject for a period of about 12 months, about 13 months, about 14 months, about 15 months, about 16 months, about 17 months, about 18 months, about 19 months, about 20 months, about 21 months, about 22 months, about 23 months, about 24 months, about 25 months, about 26 months, about 27 months, about 28 months, about 29 months, or about 30 months. In some embodiments, the BTK inhibitor is administered to the subject for a period of about 24 months. In some embodiments, the BTK inhibitor is administered to the subject concurrent with the use of immunosuppressive therapy or immunomodulatory therapy. In some embodiments, the BTK inhibitor is administered to the subject for a period of about 1 to about 2 years after initiation of immunosuppressive therapy.

Described are methods of preventing or reducing the risk of developing an EBV-positive LPD in a subject having an autoimmune disorder comprising administering to the subject a pharmaceutical composition comprising an effective dose of a BTK inhibitor. Lymphoproliferative diseases that can be prevented, or for which the risk can be reduced, include, but are not limited to, iatrogenic lymphoproliferative disorder, post-transplant lymphoproliferative disorder (PTLD), Burkitt lymphoma, B-cell non-Hodgkin lymphoma, diffuse large B-cell lymphoma (DLBCL), CNS lymphoma, NK/T-cell lymphoma, T cell non-Hodgkin lymphoma, Hodgkin lymphoma, nasopharyngeal cancer, oral hairy leukoplakia (in HIV positive patients), and an AIDS-related lymphomas. The autoimmune disorder can be, but is not limited to, lupus, systemic lupus erythematosus, rheumatoid arthritis, myelodysplastic syndromes (MDS), inflammatory bowel disease, Crohn's disease, ulcerative colitis, multiple sclerosis, aplastic anemia, alopecia areata, or HIV/AIDS, or a subject being treating with immunosuppressive or immunomodulatory therapy, such as T cell suppressive therapy or anti-TNF alpha therapy. T cell suppressive therapy can be, but is not limited to, antithymocyte globulin or recombinant antithymocyte globulin (ATG or rATG) therapy.

Also described are methods of treating oral hairy leukoplakia (OHL) in a subject having HIV/AIDS, the methods comprising administering to the subject a pharmaceutical composition comprising an effective dose of a Bruton's tyrosine kinase (BTK) inhibitor. The pharmaceutical composition can be administered to the subject upon diagnosis of OHL and can be continued for as long as the OHL condition persists

In some embodiments, methods are described for preventing or reducing the risk of developing EBV-LPD in a subject having HIV/AIDS, the methods comprising administering to the subject a BTK inhibitor. In some embodiments, the subject has poorly controlled HIV/AIDS. In some embodiments, the EBV-lymphoma or EBV-cancer is iatrogenic lymphoproliferative disorder, Burkitt lymphoma, B-cell non-Hodgkin lymphoma, diffuse large B-cell lymphoma (DLBCL), CNS lymphoma, NK/T-cell lymphoma, T cell non-Hodgkin lymphoma, Hodgkin lymphoma, nasopharyngeal cancer, oral hairy leukoplakia, and an AIDS-related lymphomas. The BTK inhibitor can be administered to the subject upon recognition or poorly controlled HIV/AIDS and can be continued for as long as the poorly controlled HIV/AIDS condition persists.

Described are methods of decreasing cleavage of procaspase 1 to caspase 1 in EBV infected cells in a subject comprising administering to the subject a pharmaceutical composition comprising an effective dose of a Bruton's tyrosine kinase (BTK) inhibitor. Cleavage of procaspase 1 to caspase 1 is a marker of inflammasome activation. Decreasing cleavage of procaspase 1 to caspase 1 can lead reduced reactivation of EBV and or reduced risk of development of EBV lymphoproliferative diseases. The subject can have a latent EBV infection. In some embodiments, the subject has been immunosuppressed, In some embodiments, the subject is receiving or has recently received immunosuppressive therapy. The subject may have or been diagnosed with a lymphoproliferative disease or an autoimmune disorder or disease. The subject may have received a transplant.

### III. Btk Inhibitors.

A BTK inhibitor is a compound that inbibits activity of BTK. In some embodiments, a BTK inhibitor binds or blocks the amino acid residue (typically cysteine) at position 481 in BTK. In some embodiments a BTK inhibitor blocks B cell receptor (BCR) signaling.

A BTK inhibitor can be an irreversible BTK inhibitor (e.g., ibrutinib, evobrutinib, and tolebrutinib) or a reversible BTK inhibitor (e.g., fenebrutinib). A BTK inhibitor can be a covalent (e.g., ibrutinib) or a non-covalent (e.g., vecabrutinib) BTK inhibitor.

BTK inhibitors include, but are not limited to, ibrutinib, acalabrutinib, pirtobrutinib, zanubrutinib, tirabrutinib, evobrutinib, vecabrutinib, tolebrutinib, elsubrutinib, nemtabrutinib, fenebrutinib, rilzabrutinib, remibrutinib, Poseltinib, M7583, Orelabrutinib, Spebrutinib, Vecabrutinib, TL-895, Edralbrutinib, Luxeptinib, Spebrutinib, and HM71224.

1. Ibrutinib (CAS Number 936563-96-1) is a small molecule drug that inhibits B-cell proliferation and survival by irreversibly binding the protein Bruton's tyrosine kinase (BTK).

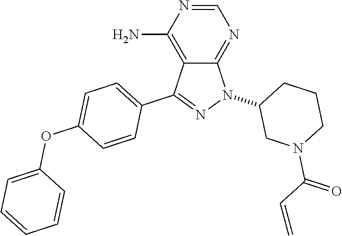

An effective dose of ibrutinib can be about 70 to about 560 mg. In some embodiments, an effective dose of ibrutinib is about 70 to about 560 mg/day. In some embodiments, an effective dose of ibrutinib is about 70 mg/day, about 140 mg/day, about 210 mg/day, about 280 mg/day, about 350 mg/day, about 420 mg/day, about 490 mg/day, or about 560 mg/day. In some embodiments, an effective dose of ibrutinib is about 35 to about 280 mg twice per day. In some embodiments, an effective dose of ibrutinib is about 35 mg, about 70 mg, about 105 mg, about 140 mg, about 175 mg, about 210 mg, about 245 mg, or about 280 mg every twelve hours.

2. Acalabrutinib (CAS Number 1420477-60-6) is a medication used to treat various types of non-Hodgkin lymphoma, including mantle cell lymphoma (MCL) and chronic lymphocytic leukemia/small lymphocytic leukemia (CLL/SLL).

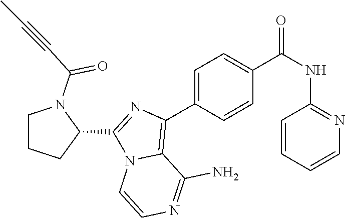 [SMILES: CC#CC(=O)N1CCC[C@H]1c1nc(-c2ccc(C(=O)Nc3ccccn3)cc2)c2c(N)nccn12]

An effective dose of acalabrutinib can be about 100 to about 400 mg. In some embodiments, an effective dose of acalabrutinib is about 100 mg/day, about 120 mg/day, about 140 mg/day, about 160 mg/day, about 180 mg/day, or about 200 mg/day. In some embodiments, an effective dose of acalabrutinib is about 50 to about 200 mg twice per day. In some embodiments, an effective dose of acalabrutinib is about 50 mg, about 60 mg, about 70 mg, about 80 mg, about 90 day, or about 100 mg every twelve hours.

### 3. Pirtobrutinib (LOXO-305) (CAS Number 2101700-15-4)

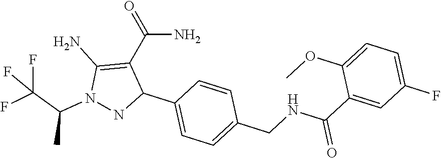 [SMILES: COc1ccc(F)cc1C(=O)NCc1ccc(C2NN([C@@H](C)C(F)(F)F)C(N)=C2C(N)=O)cc1]

An effective dose of pirtobrutinib can be about 25 to about 1000 mg. In some embodiments, an effective dose of pirtobrutinib is about 25 to about 1000 mg/day. In some embodiments, an effective dose of pirtobrutinib is about 25 mg/day, about 50 mg/day, about 100 mg/day, about 150 mg/day, about 200 mg/day, about 250 mg/day, about 300 mg/day, about 400 mg/day, about 500 mg/day, or about 1000 mg/day. In some embodiments, an effective dose of pirtobrutinib is about 200 mg/day. In some embodiments, an effective dose of pirtobrutinib is about 12.5 to about 500 mg twice per day. In some embodiments, an effective dose of pirtobrutinib is about 12.5 mg, about 25 mg, about 50 mg, about 75 mg, about 100 mg, about 125 mg, about 150 mg, about 200 mg, about 250 mg, or about 500 mg every twelve hours. In some embodiments, an effective dose of pirtobrutinib is about 100 mg every twelve hours.

### 4. Zanubrutinib (CAS Number 1691249-45-2)

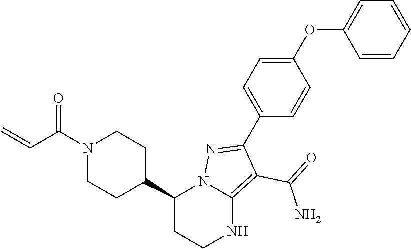 [SMILES: C=CC(=O)N1CCC([C@@H]2CCNc3c(C(N)=O)c(-c4ccc(Oc5ccccc5)cc4)nn32)CC1]

An effective dose of zanubrutinib can be about 160 to about 640 mg. In some embodiments, an effective dose of zanubrutinib is about 160 mg/day, about 240 mg/day, about 320 mg/day, about 400 mg/day, about 480 mg/day, about 560 mg/day, or about 640 mg/day. In some embodiments, an effective dose of zanubrutinib is about 80 to about 320 mg twice per day. In some embodiments, an effective dose of zanubrutinib is about 80 mg, about 120 mg, about 160 mg, about 200 mg, about 240 day, about 280 day, or about 320 mg every twelve hours.

### 5. Tirabrutinib (Velexbru, ONO/GS-4059) (CAS No 1351636-18-4)

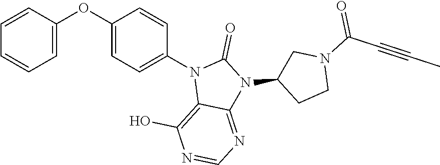 [SMILES: CC#CC(=O)N1CC[C@@H](n2c(=O)n(-c3ccc(Oc4ccccc4)cc3)c3c(O)ncnc32)C1]

An effective dose of tirabrutinib can be about 160 to about 640 mg. In some embodiments, an effective dose of tirabrutinib is about 160 mg/day, about 240 mg/day, about 320 mg/day, about 400 mg/day, about 480 mg/day, about 560 mg/day, about 600 mg/day, or about 640 mg/day. In some embodiments, an effective dose of tirabrutinib is about 80 to about 320 mg twice per day. In some embodiments, an effective dose of tirabrutinib is about 80 mg, about 120 mg, about 160 mg, about 200 mg, about 240 day, about 280 day, about 300 day, or about 320 mg every twelve hours.

### 6. Evobrutinib (CAS No. 1415823-73-2)

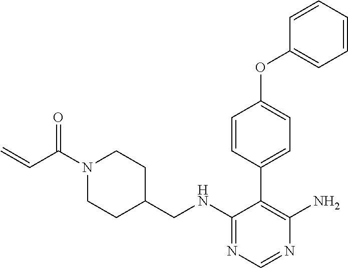 [SMILES: C=CC(=O)N1CCC(CNc2ncnc(N)c2-c2ccc(Oc3ccccc3)cc2)CC1]

### 7. Vecabrutinib (SNS-062) (CAS No.: 1510829 Jun. 7)

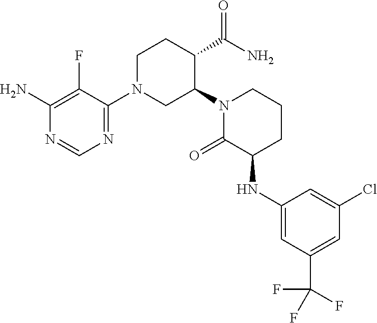 [SMILES: NC(=O)[C@H]1CCN(c2ncnc(N)c2F)C[C@@H]1N1CCC[C@@H](Nc2cc(Cl)cc(C(F)(F)F)c2)C1=O]

8. Tolebrutinib (No. CAS: 1971920-73-6)

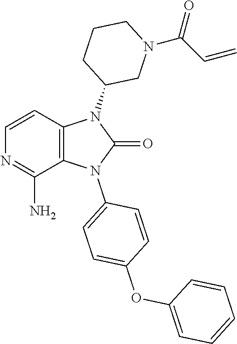 [SMILES: C=CC(=O)N1CCC[C@@H](n2c(=O)n(-c3ccc(Oc4ccccc4)cc3)c3c(N)nccc32)C1]

9. Elsubrutinib (ABBV-105) (No. CAS: 1643570-24-4)

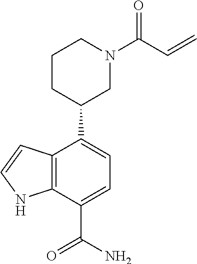 [SMILES: C=CC(=O)N1CCC[C@@H](c2ccc(C(N)=O)c3[nH]ccc23)C1]

### 10. Nemtabrutinib (ARQ 531) (CAS No. 2095393-15-8)

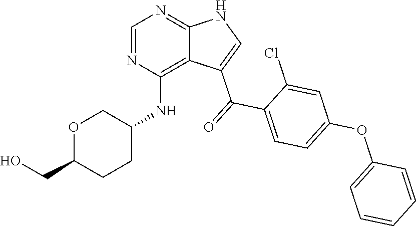 [SMILES: O=C(c1ccc(Oc2ccccc2)cc1Cl)c1c[nH]c2ncnc(N[C@@H]3CC[C@@H](CO)OC3)c12]

### 11. Fenebrutinib (CAS No. 1434048-34-6)

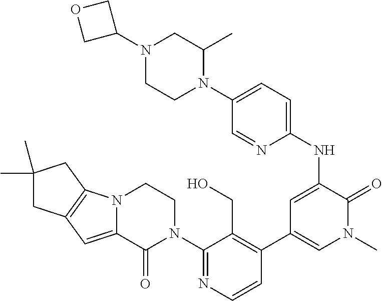

### 12. Rilzabrutinib (CAS #1575596-29-0)

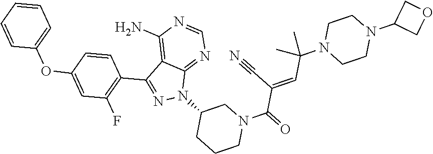 [SMILES: CC(C)(/C=C(\C#N)C(=O)N1CCC[C@H](n2nc(-c3ccc(Oc4ccccc4)cc3F)c3c(N)ncnc32)C1)N1CCN(C2COC2)CC1]

### 13. Remibrutinib (CAS No. 1787294 Jul. 8)

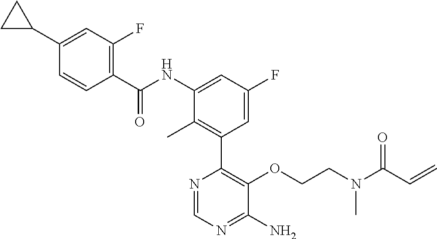 [SMILES: C=CC(=O)N(C)CCOc1c(N)ncnc1-c1cc(F)cc(NC(=O)c2ccc(C3CC3)cc2F)c1C]

Additional BTK inhibitors include, but are not limited to: Poseltinib, M7583, Orelabrutinib, Spebrutinib, Vecabrutinib, TL-895, Edralbrutinib, Luxeptinib, Spebrutinib, and HM71224.

### IV. Pharmaceutical Compositions

In some embodiments, the pharmaceutical composition comprises a dosage form suitable for administering a BTK inhibitor to a subject A pharmaceutical composition includes a pharmacologically effective amount of a BTK inhibitor as an active ingredient, and optionally one or more pharmaceutically acceptable excipients, and optionally one or more additional therapeutic agents. Pharmaceutically acceptable excipients (excipients) are substances other than the Active Pharmaceutical ingredient (API, therapeutic product) that are intentionally included in the drug delivery system. Excipients do not exert or are not intended to exert a therapeutic effect at the intended dosage. Excipients may act to a) aid in processing of the drug delivery system during manufacture, b) protect, support or enhance stability, bioavailability or patient acceptability of the API, c) assist in product identification, and/or d) enhance any other attribute of the overall safety, effectiveness, of delivery of the API during storage or use. A pharmaceutically acceptable excipient may or may not be an inert substance.

Excipients include, but are not limited to: absorption enhancers, anti-adherents, anti-foaming agents, anti-oxidants, binders, bulking agents, buffering agents, carriers, coating agents, colors, delivery enhancers, delivery polymers, dextran, dextrose, diluents, disintegrants, emulsifiers, extenders, fillers, flavors, glidants, humectants, lubricants, oils, polymers, preservatives, saline, salts, solvents, sugars, suspending agents, sustained release matrices, sweeteners, thickening agents, tonicity agents, vehicles, water-repelling agents, and wetting agents.

The carrier can be, but is not limited to, a solvent or dispersion medium containing, for example, water, ethanol, polyol (for example, glycerol, propylene glycol, and liquid polyethylene glycol), and suitable mixtures thereof. A carrier may also contain adjuvants such as preservatives, wetting agents, emulsifying agents and dispersing agents. A carrier may also contain isotonic agents, such as sugars, polyalcohols, sodium chloride, and the like.

The pharmaceutical compositions can contain other additional components commonly found in pharmaceutical compositions. Such additional components can include, but are not limited to, anti-pruritics, astringents, local anesthetics, or anti-inflammatory agents (e.g., antihistamine, diphenhydramine, etc.).

Pharmaceutically acceptable refers to those properties and/or substances which are acceptable to the subject from a pharmacological/toxicological point of view. The phrase pharmaceutically acceptable refers to molecular entities, compositions, and properties that are physiologically tolerable and do not typically produce an allergic or other untoward or toxic reaction when administered to a subject. In some embodiments, a pharmaceutically acceptable compound is approved by a regulatory agency of the Federal or a state government or listed in the U.S. Pharmacopeia or other generally recognized pharmacopeia for use in animals and more particularly in humans.

In some embodiments, the pharmaceutical composition comprises a dosage form containing dosage levels adjusted appropriately for administration to children.

In some embodiments, the pharmaceutical composition further comprises one or more additional therapeutics.

The described pharmaceutical compositions can be formulated as liquid formulations or as solid formulations (including powders or lyophilized formulations). The described pharmaceutical compositions can be provided as, for example, a powder, granule, liquid, aqueous solution, suspension, cream, ointments, or pill. Pills include, but are not limited to, lozenges, capsules, tablets, and caplets. A pharmaceutical composition suitable for oral administration can be provided as a powder, granule, liquid, suspension, or pill. The described pharmaceutical compositions may be provided in extending release formulations or a bolus formulations.

The described pharmaceutical compositions can be formulated for repeat dosing. The described pharmaceutical compositions can be formulated for administration once per day, twice per pay, three times per day, four times per day, or every 3, 4, 6, 8, or 12, or 24 hours. The described pharmaceutical compositions may be provided in unit-dose or multi-dose containers or pills.

### V. Administration

The pharmaceutical compositions can be administered to a subject via enteral, parenteral, inhalational, transdermal, transmucosal, sublingual, buccal, and topical routes. Enteral administration includes, but is not limited to, oral, gastric or duodenal feeding tube, rectal suppository, and rectal enema. Parenteral administration includes, but is not limited to, injection, infusion, intraarterial, intracardiac, intradermal, intraduodenal, intramedullary, intramuscular, intraosseous, intraperitoneal, intrathecal, intravascular, intravenous, intravitreal, epidural, and subcutaneous administration. Topical administration includes, but is not limited to, epicutaneous, dermal, enema, eye drops, ear drops, intranasal, vaginal administration. In some embodiments, the pharmaceutical compositions are formulated for oral delivery.

The described pharmaceutical compositions are administered in an amount effective for inhibiting activation of EBV or inhibiting switching of EBV from latent to lytic phase. An effective amount can be an amount that is capable of at least partially preventing or reducing the risk of developing a lymphoproliferative disease. The dose required to obtain an effective amount may vary depending on the agent, formulation, and individual to whom the agent is administered. Determination of an effective amounts may involve an in vitro assay in which varying doses of agent are administered to cells in culture and the concentration of agent effective for ameliorating some or all symptoms is determined in order to calculate the concentration required in vivo. Determination of an effective amounts may be based on in vivo animal studies.

### VI. Kits

In some embodiments, kits containing the described pharmaceutical compositions are described. The kits comprises the BTK inhibitor and instructions for administering and/or treating a subject, such as an immunosuppressed subject. In some embodiments, the kit comprises ibrutinib and instructions for administering and/or treating an immunosuppressed subject. Instructions can include documents describing relevant materials or methodologies pertaining to the kit. The instructions may include one or more of: background information, list of components and their availability information (purchase information, etc.), brief or detailed protocols for using the kit, trouble-shooting guidance, references, technical support, indications, usage, dosage, administration, contraindications and/or warnings concerning the use the drug, and any other related documents. Instructions can be supplied with the kit or as a separate member component, either as a paper form or an electronic form. The instructions may include a notice in a form prescribed by a governmental agency regulating the manufacture, use or sale of pharmaceuticals or biological products, which notice reflects approval by the agency of manufacture, use or sale for human administration. A kit can be manufactured as a single use unit dose for one subject, multiple uses for a particular subject. The kit components may be assembled in cartons, blister packs, bottles, tubes, and the like.

It is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

### EXAMPLES

### Example 1. Ibrutinib Treatment Inhibits EBV Lytic Cycle

EBV-positive HH514-16 Burkitt lymphoma cell line was exposed to varying concentrations of the BTK inhibitor Ibrutinib for 2 hours before treatment with the lytic cycle inducing agent sodium butyrate (NaB; an HDAC inhibitor) for 24 or 48 hours. Cells were harvested, fixed and permeabilized, stained with a monoclonal antibody to the viral latent-to-lytic switch protein ZEBRA (Z EBV Replication Activator), and examined by flow cytometry. ZEBRA is an early lytic protein of EBV, encoded by BZLFI, that drives the lytic cascade. Cells were stained in parallel with an isotype-matched control antibody for placement of gates. The results show that treatment of cells with the BTK inhibitor ibrutinib resulted in decreased levels of ZEBRA, indictive of inhibition of EBV switching to lytic phase (Table 1). Decreasing EBV switching to lytic phase decreases the risk of the B cells developing lymphoma.

TABLE 1
Percent ZEBRA positive HH514-16 Burkitt lymphoma treated
with ibrutinib and inducing agent NaB.

| | | Induction | | | |
|---|---|---|---|---|---|
| | Lytic phase | time | | Percent positive cells | |
| Cell type | inducer | (h) | Ibrutinib | Control | ZEBRA ^{a} |
| HH514-16 | Sodium | 24 | 0 | 0.1 | 26 |
| | Butyrate | | 0.25 μM | 0.1 | 13 |
| | | | 0.5 μM | 0.1 | 9.5 |
| | | | 1 μM | 0.1 | 9.0 |
| | | | 2 μM | 0.1 | 8.6 |
| | | | 5 μM | 0.1 | 7.0 |
| HH514-16 | Sodium | 48 | 0 | 0.1 | 45 |
| | Butyrate | | 0.25 μM | 0.1 | 38 |
| | | | 0.5 μM | 0.1 | 30 |
| | | | 1 μM | 0.1 | 23 |
| | | | 2 μM | 0.1 | 22 |
| | | | 5 μM | 0.1 | 18 |

^{a} marker of lytic phase

### Example 2. Ibrutinib Treatment Reduces Expression of EBV Lytic Cycle Genes

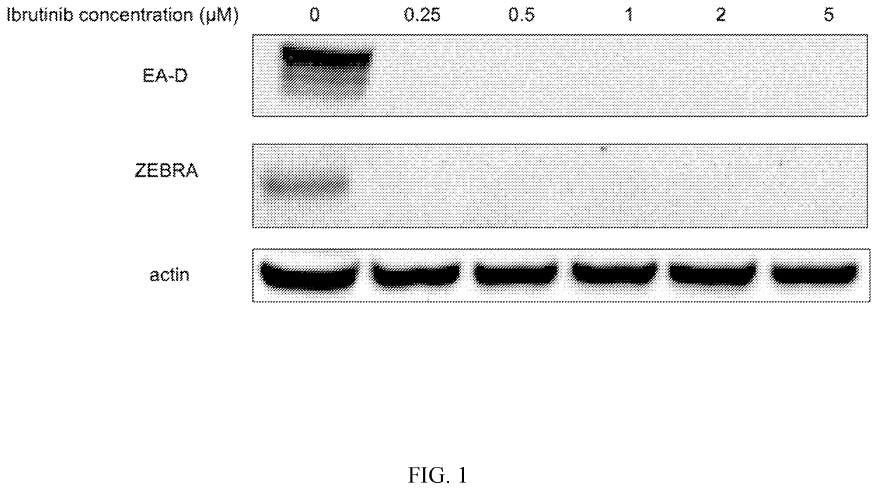

EBV-positive HH514-16 Burkitt lymphoma cell line was exposed to varying concentrations of the BTK inhibitor Ibrutinib for 2 hours before treatment with the lytic cycle inducing agent sodium butyrate (NaB; an HDAC inhibitor) for 24 or 48 hours. Cells were harvested and subjected to immunoblotting with monoclonal antibodies to ZEBRA and EA-D (Early antigen-diffuse). EA-D is useful in the diagnosis of acute Epstein-Barr virus (EBV) infection and EBV reactivation and functions downstream of ZEBRA. As shown in FIG. 1, expression of ZEBRA and EA-D were significantly reduced in cells treated with Ibrutinib prior to exposure to lytic cycle inducing agent NaB.

### Example 3. Ibrutinib Treatment Inhibits EBV Lytic Cycle

EBV-positive HH514-16 Burkitt lymphoma cell line was exposed to varying concentrations of the BTK inhibitor Ibrutinib for 2 hours before treatment with the lytic cycle inducing agent azacytidine (AZA; 5-aza-2′-deoxycytidine, a DNA methyltransferase inhibitor) for 24 or 48 hours. Cells were harvested, fixed and permeabilized, stained with a monoclonal antibody to the viral latent-to-lytic switch protein ZEBRA (Z EBV Replication Activator), and examined by flow cytometry. Cells were stained in parallel with an isotype-matched control antibody for placement of gates. The results show that treatment of cells with the BTK inhibitor ibrutinib resulted in decreased levels of ZEBRA following exposure to the inducing agent AZA (Table 2). Ibrutinib effectively decreases EBV lytic cycle in response to two different inducers, AZA and NaB (Example 1, Table 1).

TABLE 2
Percent ZEBRA positive HH514-16 Burkitt lymphoma treated
with ibrutinib and inducing agent AZA.

| | | Induction | | | |
|---|---|---|---|---|---|
| | Lytic phase | time | | Percent positive cells | |
| Cell type | inducer | (h) | Ibrutinib | Control | ZEBRA ^{a} |
| HH514-16 | AZA | 24 | 0 | 0.1 | 18 |
| | | | 0.25 μM | 0.1 | 13 |
| | | | 0.5 μM | 0.1 | 11 |
| | | | 1 μM | 0.1 | 9.0 |
| | | | 2 μM | 0.1 | 10 |
| | | | 5 μM | 0.1 | 9.0 |
| HH514-16 | AZA | 48 | 0 | 0.1 | 27 |
| | | | 0.25 μM | 0.1 | 13 |
| | | | 0.5 μM | 0.1 | 13 |
| | | | 1 μM | 0.1 | 15 |
| | | | 2 μM | 0.1 | 13 |
| | | | 5 μM | 0.1 | 14 |

^{a} marker of lytic phase

### Example 4. Ibrutinib Treatment Reduces Expression of EBV Lytic Cycle Genes

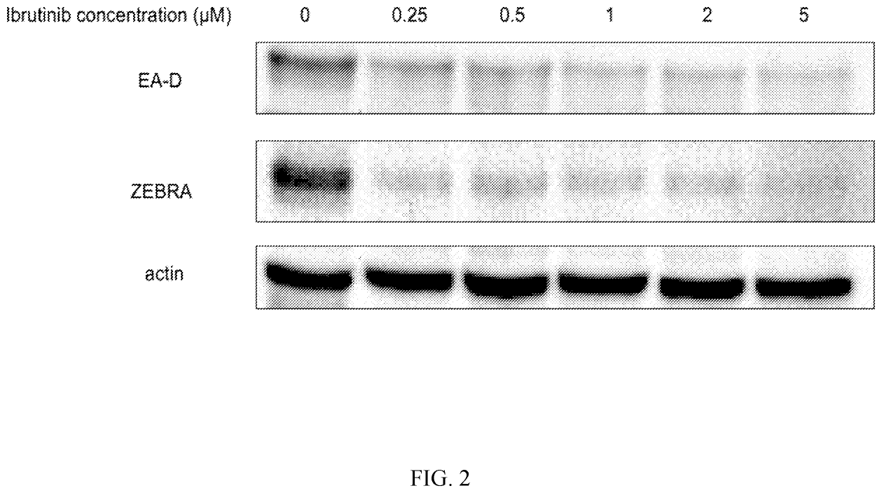

EBV-positive HH514-16 Burkitt lymphoma cell line was exposed to varying concentrations of the BTK inhibitor Ibrutinib for 2 hours before treatment with the lytic cycle inducing agent azacytidine (AZA; 5-aza-2′-deoxycytidine, a DNA methyltransferase inhibitor) for 24 hours. Cells were harvested and subjected to immunoblotting with monoclonal antibodies to ZEBRA and EA-D (Early antigen-diffuse). As shown in FIG. 2, expression of ZEBRA and EA-D were significantly reduced in cells treated with Ibrutinib prior to exposure to lytic cycle inducing agent AZA.

### Example 5. Ibrutinib Treatment Inhibits EBV Lytic Cycle in Akata Cells

EBV-positive Akata Burkitt lymphoma cell line was exposed to varying concentrations of the BTK inhibitor Ibrutinib for 2 hours before treatment with rabbit-anti human IgG (a PI3K inhibitor) that activates the EBV lytic cycle for 24 hours. Akata cells are an EBV-producer line established from a Japanese patient with Burkitt's lymphoma. Akata cells produce transforming virus upon treatment of cells with anti-immunoglobulin antibodies. Cells were harvested, fixed and permeabilized, stained with a monoclonal antibody to the viral latent-to-lytic switch protein ZEBRA (Z EBV Replication Activator), and examined by flow cytometry. Cells were stained in parallel with an isotype-matched control antibody for placement of gates. The results show that treatment of cells with the BTK inhibitor ibrutinib resulted in decreased levels of ZEBRA in Akata cells following exposure to the inducing agent IgG (Table 3). Ibrutinib effectively decreases EBV lytic cycle in two different cell types (HH514-16 and Akata) in response to three different inducers, NaB, AZA, and IgG that act in different pathways in inducing EBV reactivation.

TABLE 3
Percent ZEBRA positive Akata cells treated with ibrutinib
and inducing agent IgG.

| | | Induction | | | |
|---|---|---|---|---|---|
| | Lytic phase | time | | Percent positive cells | |
| Cell type | inducer | (h) | Ibrutinib | Control | ZEBRA ^{a} |
| Akata | Rabbit anti- | 24 | 0 | 2.0 | 14 |
| | human IgG | | 0.25 μM | 2.0 | 7 |
| | | | 0.5 μM | 2.0 | 7 |
| | | | 1 μM | 2.0 | 5.5 |
| | | | 2 μM | 2.0 | 5.7 |
| | | | 5 μM | 2.0 | 5.7 |

^{a} marker of lytic phase

### Example 6. Ibrutinib Treatment Reduces Expression of EBV Lytic Cycle Genes

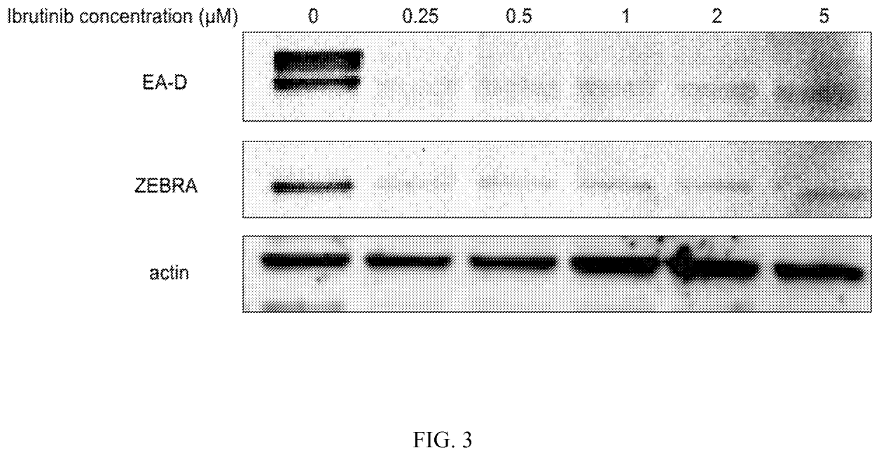

EBV-positive Akata Burkitt lymphoma cell line was exposed to varying concentrations of the BTK inhibitor Ibrutinib for 2 hours before treatment with anti-IgG molecules that activate the EBV lytic cycle for 24 hours. Cells were harvested and subjected to immunoblotting with monoclonal antibodies to ZEBRA and EA-D (Early antigen-diffuse). As shown in FIG. 3, expression of ZEBRA and EA-D were significantly reduced in cells treated with Ibrutinib prior to exposure to lytic cycle inducing agent IgG.

### Example 7. BTK Inhibitors Reduce Expression of ZEBRA in HH514-16 Burkitt Lymphoma Cells

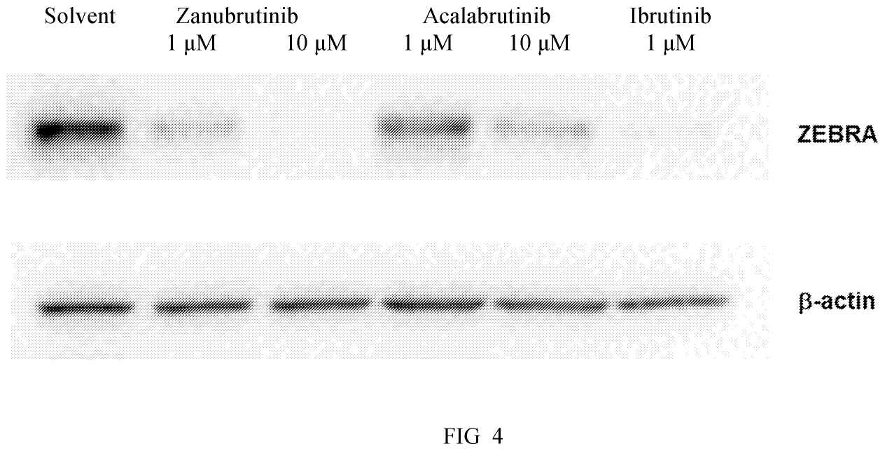

A) EBV-positive HH514-16 Burkitt lymphoma cells were exposed to varying concentrations of the BTK inhibitors Zanubrutinib, Acalabrutinib, or Ibrutinib for 2 hours before treatment with sodium butyrate for 24 hours to activate the EBV lytic cycle. Cells were harvested and subjected to immunoblotting with monoclonal antibodies to ZEBRA or the control protein β-actin to analyze protein expression. As shown in FIG. 4, expression of ZEBRA was significantly reduced in cells treated with Zanubrutinib, Acalabrutinib, or Ibrutinib prior to induction of the EBV lytic cycle.

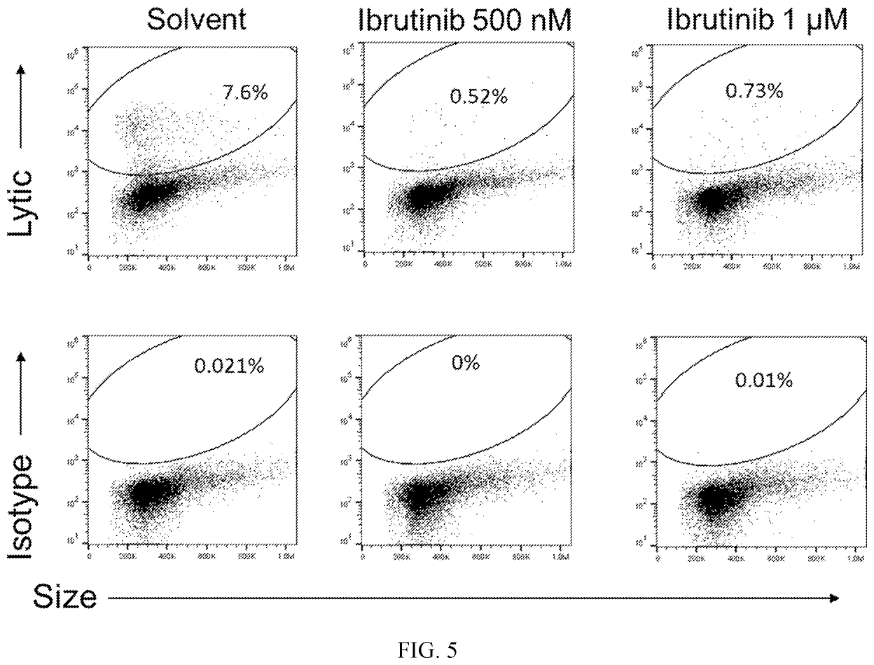

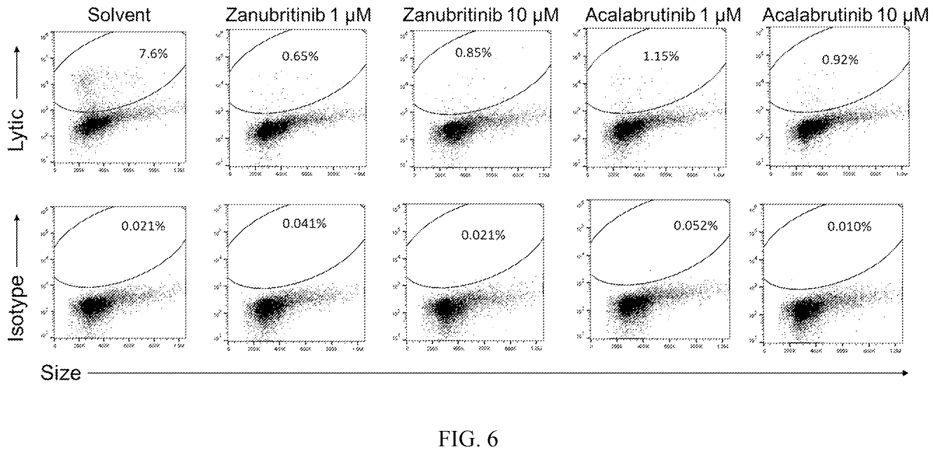

B) EBV-positive Mutu-I Burkitt lymphoma cells were exposed to varying concentrations of the BTK inhibitors Zanubrutinib, Acalabrutinib, or Ibrutinib for 2 hours before treatment with sodium butyrate for 24 hours to activate the EBV lytic cycle. Cells were harvested, fixed and permeabilized, stained with a monoclonal antibody to the viral latent-to-lytic switch protein ZEBRA, and examined by flow cytometry. Cells were stained in parallel with an isotype-matched control antibody for placement of gates. As shown in FIGS. 5 and 6 and Table 4, treatment of cells with the BTK inhibitors resulted in decreased levels of ZEBRA, indictive of inhibition of EBV switching to lytic phase. Decreasing EBV switching to lytic phase decreases the risk of the B cells developing lymphoma.

TABLE 4
Percent ZEBRA positive Mutu-I Burkitt lymphoma
cells treated with the indicated BTK inhibitor and
induced with sodium butyrate for 24 hours.

| | | Percent |
|---|---|---|
| | Inhibitor | ZEBRA |
| | conc. | positive |
| BTK inhibitor | [μM] | cells |
| none | 0 | 7.6 |
| Ibrutinib | 0.5 | 0.52 |
| Ibrutinib | 1 | 0.73 |
| Zanubrutinib | 1 | 0.65 |
| Zanubrutinib | 10 | 0.85 |
| Acalabrutinib | 1 | 1.15 |
| Acalabrutinib | 10 | 0.92 |

### Example 8. Affect of BTK Inhibitors on ZEBRA, Procaspase 1, and Cleaved Caspase 1 in Burkitt Lymphoma Cells

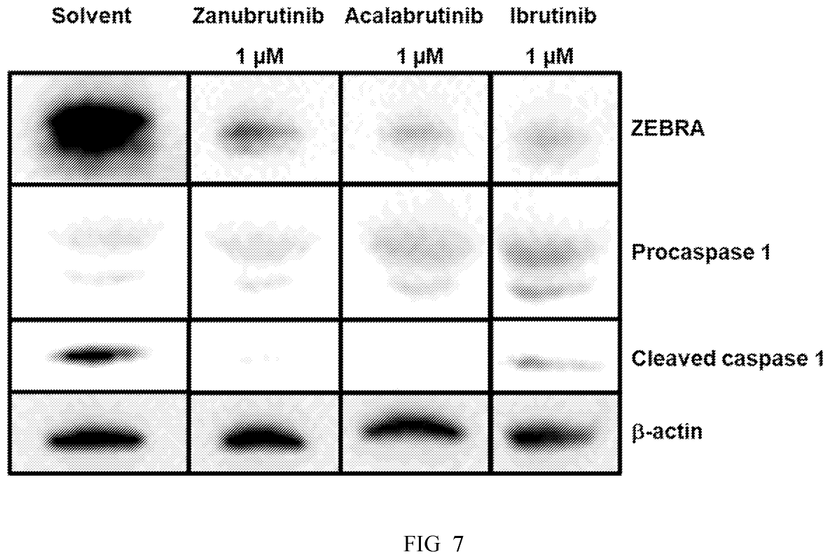

EBV-positive Mutu-I Burkitt lymphoma cells were exposed to 1 μM Zanubrutinib, Acalabrutinib, or Ibrutinib for 2 hours before treatment with sodium butyrate for 24 hours to activate the EBV lytic cycle. Cells were harvested and subjected to immunoblotting with monoclonal antibodies to ZEBRA, procaspase 1, cleaved caspase 1, or the control protein β-actin to analyze protein expression. As shown in FIG. 7, expression of ZEBRA was significantly reduced in cells treated with Zanubrutinib, Acalabrutinib, or Ibrutinib prior to induction of the EBV lytic cycle. Cleaved caspase 1 was also significantly reduced in cells treated with Zanubrutinib, Acalabrutinib, or Ibrutinib prior to induction of the EBV lytic cycle. Cleavage of caspase is a marker for programmed cell death. Decreased levels of caspase indicate increased survival of the B cells when treated with Zanubrutinib, Acalabrutinib, or Ibrutinib.

1. A method of decreasing Epstein-Barr Virus (EBV) lytic cycle in an immunosuppressed subject comprising administering to the subject a pharmaceutical composition comprising an effective dose of a Bruton's tyrosine kinase (BTK) inhibitor.

2. A method of preventing or reducing the risk of developing an EBV-positive lymphoproliferative disease (LPD), EBV-associated malignancy or pre-malignancy in an immunosuppressed subject comprising administering to the subject a pharmaceutical composition comprising an effective dose of a BTK inhibitor.

3. The method of claim 1, wherein the BTK inhibitor is an irreversible BTK inhibitor.

4. The method of claim 1, wherein the BTK inhibitor is a reversible BTK inhibitor.

5. The method of claim 1, wherein the BTK inhibitor is selected from the group consisting of: ibrutinib, acalabrutinib, zanubrutinib, pirtobrutinib, tirabrutinib, evobrutinib, vecabrutinib, tolebrutinib, elsubrutinib, nemtabrutinib, fenebrutinib, rilzabrutinib, remibrutinib, Poseltinib, M7583, Orelabrutinib, Spebrutinib, Vecabrutinib, TL-895, Edralbrutinib, Luxeptinib, Spebrutinib, and HM71224.

6. The method of claim 1, wherein the BTK inhibitor comprises ibrutinib, acalabrutinib, or zanubrutinib.

7. The method of claim 2, wherein the LPD is a iatrogenic lymphoproliferative disorder, a post-transplant lymphoproliferative disorder, a Burkitt lymphoma, a B-cell non-Hodgkin lymphoma, a diffuse large B-cell lymphoma, a CNS lymphoma, a NK/T-cell lymphoma, a T cell non-Hodgkin lymphoma, a Hodgkin lymphoma, a nasopharyngeal cancer, a gastric cancer, a oral hairy leukoplakia, or an AIDS-related lymphoma.

8. The method of claim 1, wherein the subject is on immunosuppressive or immunomodulatory therapy.

9. The method of claim 8, wherein the immunosuppressive or immunomodulatory therapy comprises T cell suppressive therapy or anti-TNF alpha therapy.

10. The method of claim 1, wherein the subject is an organ transplant recipient or a hematopoietic transplant recipient.

11. The method of claim 10, wherein the subject is seronegative for EBV at the time of the transplant, has an active or suspected active EBV infection at the time of or following the transplant, is EBV positive and receives an EBV-negative donor organ or hematopoietic transplant, has a high degree of HLA mismatch with the donor, has an active or suspected active CMV infection at the time of transplantation, is CMV positive and receives a CMV-negative donor organ or hematopoietic transplant, has or is suspected of having acute or chronic graft versus host disease, is at risk of acute or chronic graft versus host disease, suffers from or is suspected of suffering from T-cell depletion, has diabetes, or is on an intense immunosuppressive drug regimen, or combination of one of more of the above.

12. The method of claim 1, wherein the subject has not been diagnosed with a LPD.

13. The method of claim 8, wherein administering the pharmaceutical composition comprises administering a first dose of the pharmaceutical composition to the subject prior to initiation of the immunosuppressive or immunomodulatory therapy, at the time of initiation of the immunosuppressive or immunomodulatory therapy, or within about 2-8 weeks of initiation of the immunosuppressive or immunomodulatory therapy.

14. The method of claim 10, wherein administering the pharmaceutical composition comprises administering a first dose of the pharmaceutical composition to the subject prior to the transplant, at the time of the transplant, or within about 2-8 weeks of the transplant.

15. The method of claim 13, wherein the pharmaceutical composition is administered to the subject for a period of about 1 to about 2 years.

16. The method of claim 1, wherein EBV levels in the subject are monitored and the pharmaceutical composition is administered to the subject if the levels of EBV in the subject increase.

17. The method of claim 1, wherein the subject has an autoimmune condition.

18. The method of claim 17, wherein the autoimmune condition is selected from the group consisting of: lupus, systemic lupus erythematosus, rheumatoid arthritis, myelodysplastic syndromes, inflammatory bowel disease, Crohn's disease, ulcerative colitis, multiple sclerosis, aplastic anemia, alopecia areata, or HIV/AIDS.

19. A method for treating oral hairy leukoplakia (OHL) in a subject having HIV/AIDS, the methods comprising administering to the subject a pharmaceutical composition comprising an effective dose of a BTK inhibitor.

20. The method of claim 19, wherein the pharmaceutical composition is administered to the subject upon diagnosis of OHL and is continued for as long as the OHL condition persists.

21. (canceled)

22. (canceled)

23. (canceled)
